(19) United States
(12) Patent Application Publication
(10) Pub. No.: US 2012/0203067 A1
(43) Pub. Date: Aug. 9, 2012

(54) METHOD AND DEVICE FOR DETERMINING THE LOCATION OF AN ENDOSCOPE

(75) Inventors: William E. Higgins, State College, PA (US); Jason D. Gibbs, State College, PA (US); Duane C. Cornish, State College, PA (US)

(73) Assignee: The Penn State Research Foundation, University Park, PA (US)

(21) Appl. No.: 13/362,123

(22) Filed: Jan. 31, 2012

Related U.S. Application Data
(60) Provisional application No. 61/439,529, filed on Feb. 4, 2011.

(51) Int. Cl. A61B 1/267; A61B 1/00

(52) U.S. Cl. 600/117

(57) ABSTRACT

A technician-free strategy enables real-time guidance of bronchoscopy. The approach uses measurements of the bronchoscope's movement to predict its position in 3D virtual space. To achieve this, a bronchoscope model, defining the device's shape in the airway tree to a given point p, provides an insertion depth to p. In real time, the invention compares an observed bronchoscope insertion depth and roll angle, measured by an optical sensor, to precalculated insertion depths along a predefined route in the virtual airway tree to predict a bronchoscope's location and orientation.

### REFERENCE TO RELATED APPLICATION

This application claims priority from U.S. Provisional Patent Application Ser. No. 61/439,529, filed Feb. 4, 2011, the entire content of which is incorporated herein by reference.

### GOVERNMENT SPONSORSHIP

This invention was made with government support under NM Grant Nos. R01-CA074325 and R01-CA151433 awarded by the National Cancer Institute. The government has certain rights in the invention.

### FIELD OF THE INVENTION

This invention relates generally to image-guided endoscopy and, in particular, to a system and method wherein real-time measurements of actual instrument movements are compared in real-time to precomputed insertion depth values based upon shape models, thereby providing continuous prediction of the instrument's location and orientation and technician-free guidance irrespective of adverse events.

### BACKGROUND OF THE INVENTION

Bronchoscopy is a procedure whereby a flexible instrument with a camera on the end, called a bronchoscope, is navigated through the body's tracheobronchial airway tree. Bronchoscopy enables a physician to perform biopsies or deliver treatment [39]. This procedure is often performed for lung cancer diagnosis and staging. Before a bronchoscopy takes place, a 3D multidetector computed tomography (MDCT) scan is created of the patient's chest consisting of a series of two-dimensional (2D) images [15, 38, 5]. A physician then uses the MDCT scan to identify a region of interest (ROI) he/she wishes to navigate to. ROIs may be lesions, lymph nodes, treatment delivery sites, lavage sites, etc. Next, either a physician plans a route to each ROI by looking at individual 2D MDCT slices or automated methods compute routes to each ROI [6, 8]. Later, during bronchoscopy, the physician attempts to maneuver the bronchoscope to each ROI along its pre-defined route. Upon reaching the planned destination, there is typically no visual indication that the bronchoscope is near the ROI, as the ROI often resides outside of the airway tree (extraluminal), while the bronchoscope is inside the airway tree (endoluminal). Because of the challenges in standard bronchoscopy, physician skill levels vary greatly, and navigation errors occur as early as the second airway generation [6, 31].

With the advances in computers, researchers are developing image-guided intervention (IGI) systems to help guide physicians during surgical procedures [11, 32, 37, 27]. Bronchoscopy-guidance systems are IGI systems that provide navigational instructions to guide a physician maneuvering a bronchoscope to an ROI [8, 4, 3, 24, 35, 14, 2, 9, 33, 30, 13, 36, I]. In order to explain how these systems provide navigational instructions, it is necessary to formally define the elements involved. The patient's chest, encompassing the airway tree, vasculature, lungs, ribs, etc., makes up the physical space. During standard bronchoscopy, two different data manifestations of the physical space are created (FIG. 1). The first data manifestation, referred to as the virtual space, is the MDCT scan. The 3D MDCT scan gives a digital representation of the patient's chest. Automated algorithms process the MDCT scan to derive airway-tree surfaces and centerlines, diagnostic ROIs, and optimal paths reaching each ROI [8, 10]. A virtual camera C_{V} placed in the derived airway tree generates endoluminal renderings (also referred to as virtual-bronchoscopy (VB) views) I_{V} [12].

The second data manifestation created during live bronchoscopy, referred to as the real space, consists of the bronchoscope camera's live stream of video frames depicting the real world from within the patient's airway tree. Each live video frame, referred to as I_{R}, represents a view from the real camera C_{R}.

To provide navigational instructions, the bronchoscopy-guidance system attempts to place C_{V} in virtual space in an orientation roughly corresponding to C_{R} in physical space. If a bronchoscopy-guidance system can do this correctly, the views, I_{V} and I_{R}, produced by C_{V} and C_{R}, are said to be synchronized. With synchronized views, the guidance system can then relate navigational information that exists in the virtual space to the physician, ultimately providing guidance to reach an ROI.

Currently, bronchoscopy guidance systems fall under two categories based on the synchronization method for I_{V} and I_{R}:1) electromagnetic navigation bronchoscopy (ENB); and 2) image-based bronchoscopy [3, 24, 35, 14, 2, 9, 13, 36, 29, 34, 28, 26, 40]. ENB systems track the bronchoscope through the patient's airways by affixing an electromagnetic (EM) sensor to the bronchoscope and generating an EM field through the patient's body [2, 9, 36, 28, 40]. As the sensor is maneuvered through the lungs, the ENB system reports its position within the EM field in real time. Image-based bronchoscopy systems derive views from the MDCT data and compare them to live bronchoscopic video using image-based registration and tracking techniques [3, 24, 35, 14, 13, 29, 34, 28, 26]. In both cases, VB views are displayed to provide guidance. Both ENB and image-based bronchoscopy methods have shortcomings that prevent continuous robust synchronization. ENB systems suffer from patient motion (breathing, coughing, etc.), electromagnetic signal noise, and require expensive equipment. Image-based bronchoscopy techniques rely on the presence of adequate information in the bronchoscope video frames to enable registration. Often times, video frames lack enough structural information to allow for image-based registration or tracking. For example, the camera C_{R} may be occluded by blood, mucous, or bubbles. Other times, C_{R} may be pointed directly at an airway wall. Because registration and tracking techniques are not robust to these events, an attending technician is required to operate the system.

### SUMMARY OF THE INVENTION

This invention overcomes the drawbacks of electromagnetic navigation bronchoscopy (ENB) and image-based bronchoscopy systems by comparing real-time measurements of actual instrument movements to precomputed insertion depth values provided by shape models. The preferred methods implement this comparison in real-time, providing continuous prediction of the instrument's tip location and orientation. In this way, the invention enables technician-free guidance and continuous procedure guidance irrespective of adverse events.

A method of determining the location of an endoscope within a body lumen according to the invention comprises the step of precomputing a virtual model of an endoscope that approximates insertion depths at a plurality of view sites along a predefined path to a region of interest (ROI). A “real” endoscope is provided with a device such as an optical sensor to observe actual insertion depths during a live procedure. The observed insertion depths are compared in real time to the precomputed insertion depths at each view site along the predefined path, enabling the location of the endoscope relative to the virtual model to be predicted at each view site by selecting the view site with the precomputed insertion depth that is closest to the observed insertion depth. An endoluminal rendering may then be generated providing navigational instructions based upon the predicted locations. The lumen may form part of an airway tree, and the endoscope may be a bronchoscope.

The device operative to observe actual insertion depths may additionally be operative to observe roll angle, which may be used to rotate the default viewing direction at a selected view site. The method of Gibbs at al. may be used to predetermine the optimal path leading to an ROI. The method may further include the step of displaying the rendered predicted locations and actual view sites from the device. The virtual model may be a MDCT image-based shape model, and the precomputing step may allow for an inverse lookup of the predicted locations. The method may include the step of calculating separate insertion depths to each view site along the medial axes of the lumen, and the endoscope may be approximated as a series of line segments.

In accordance with certain preferred embodiments, the lumen is defined using voxel locations, and the method may include the step of calculating separate insertion depths to any voxel location within the lumen and/or approximating the shape of the endoscope to any voxel location within the lumen. The insertion depth to each view site may be calculated by summing distances along the lumen medial axes. The insertion depth to each voxel location within the lumen may be calculated by finding the shortest distance from a root voxel location to every voxel location within the lumen using Dijkstra's algorithm, or calculated by using a dynamic programming algorithm. The shape of the endoscope may be approximated using the lumen medial axes or through the use of Dijkstra's algorithm. The edge weight used in Dijkstra's algorithm may be determined using a dot product and the Euclidean distance between voxel locations within the lumen. If utilized, the dynamic programming function may include an optimization function based on the dot product between voxel locations within the lumen.

### BRIEF DESCRIPTION OF THE DRAWINGS

FIG. 1 shows how the “real” patient establishes the physical space (left). The patient has two data manifestations created for his or her body during the bronchoscopy process: 1) Virtual Space; and 2) Real Space. The virtual space is derived from the patient's 3D MDCT scan, including virtual-bronchoscopy views rendered from within a virtual airway tree. The real-space data manifestation comprises a stream of bronchoscopic video frames provided by the bronchoscope's camera during a procedure. Bronchoscopy guidance systems register the virtual space and the real space. (The physical space representation is a drawing by Terese Winslow, Bronchoscopy, NCl Visuals Online, National Cancer Institute.);

FIG. 2 shows a block diagram of the method of the invention;

FIG. 3 shows a sensor is mounted externally to the patient's body. As the bronchoscope moves past the sensor, the sensor can collect bronchoscope insertion movements (“Y”) and roll movements (“X”);

FIGS. 4A-4C are a visualization of the three proposed bronchoscope-model types for a simple, controlled geometry created from PVC pipes. Several sample models (dark tubes), each beginning at the lower right and ending partially through the PVC pipe, appear for each type. The centerline model has no flexibility in its shape, and, hence, appears to only show one model. Each bronchoscope model represents the shape of the bronchoscope at various insertion depths;

FIG. 5 shows three schematic 2D bronchoscope models. A model gives a better solution with respect to the optimization function (8) while moving left to right. This optimization finds solutions that emulate the physical behavior of a bronchoscope;

FIG. 6A shows an airway tree depicted along with a fictional ROI (dark sphere) serving as the navigational target;

FIG. 6B shows an experimental setup displaying the airway phantom, navigational sensor, and apparatus for ground-truth roll-angle measurements. A third party used airway-surface data provided by us to construct the phantom out of a rigid thermoplastic material.

FIGS. 7A-7D show views predicted using sensor measurements compared to the corresponding bronchoscopic video frames when the bronchoscope was inserted 75 mm into the lung phantom (sensor reading=76 mm);

FIGS. 8A-8C show views of the bronchoscope model from the three different methods at the predicted view sites that are 76 mm past a registration point near the main carina; and

FIGS. 9A-9B show the worst error observed during the phantom experiment occurred at an true insertion depth of 21 mm. The mouse sensor was off by 6 mm causing the centerline model to predicte a location 7 mm short of the true bronchoscope location. The video frame from the real bronchoscope is depicted (FIG. 9A) next to the virtual view generated from the centerline model (FIG. 9B).

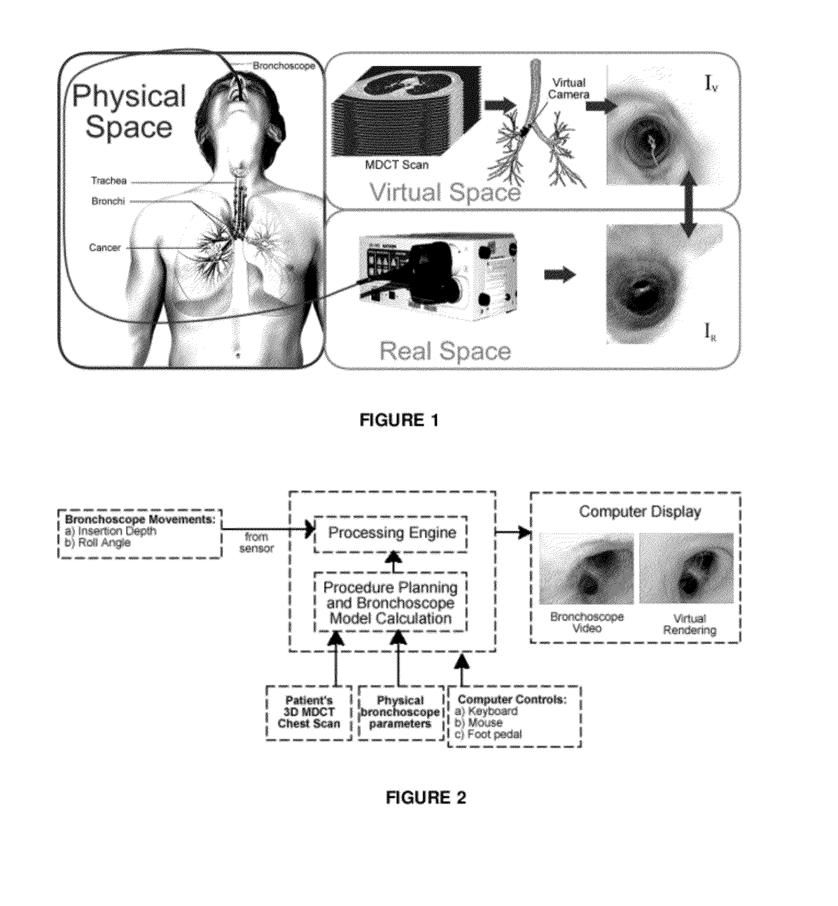

### DETAILED DESCRIPTION OF THE INVENTION

To overcome the drawbacks of ENB and image-based bronchoscopy systems, we propose a fundamentally different method. Our method compares real-time measurements of the bronchoscope movement to precomputed insertion depth values in the lungs provided by MDCT-image-based bronchoscope-shape models. Our method uses this comparison to provide a real-time, continuous prediction of the bronchoscope tip's location and orientation. In this way, our method then enables continuous procedure guidance irrespective of adverse events. It also enables technician-free guidance.

### Branching Organ Representation

Let M be a 3D MDCT scan of the patient's airway tree N. While we focus on bronchoscopy, the invention is applicable to any procedure requiring guidance though a tubular structure, such as the colon or vasculature.

A virtual N is segmented from M using the method of Graham et al. [10]. This results in a binary-valued volume:

$$v ( x , y , z ) = \{ \begin{matrix}1 , & if l is inside N \\ 0 , & otherwise\end{matrix} \qquad ( 1 )$$

representing a set of voxels V_{seg}, where v(x, y, z)εV_{seg} [symbol] v(x, y, z)=1.

Using the branching organ conventions of Kiraly et al., the centerlines of N can be derived using the method developed by Yu et al., resulting in a tree T=(V,B,P) [16, 41, 42]. V is a set of view sites {v₁, . . . vⱼ}, where J≧1 is an integer. Each view site v=(x,y,z,α,β,γ), where (x,y,z) denotes v's 3D position in M and (α,β,γ) denotes the Euler angles defining the default view direction at v. Each vεV is located on one of the centerlines of N. Therefore, V is referred to as the set of the airway tree's centerlines, and it represents the set of centralized axes that follow all possible navigable routes in N. B is a set of branches {b₁, . . . , bₖ}, where each b={v_{c}, . . . , vᵢ}, v_{c}, . . . , vᵢεV, and 0≦c≦i. Each branch must begin at either the first view site at the origin of the organ, called the root site, or at a bifurcation. Each branch must end at either a bifurcation or at any terminating view site e. A terminating view site is any view site that has no children. P is a set of paths, {p₁, . . . , pₘ}, where each p consists of connected branches. A path must begin at the root site and end at a terminating view site e.

### Bronchoscope Tracking Method

The invention comprises two major aspects (FIG. 2): 1) a computer-based prediction engine driven by a precomputed bronchoscope model; and 2) an optical sensor interfaced between a bronchoscope and a computer. The computer-generated bronchoscope model approximates the insertion depth to each view site. Before a bronchoscopy, we use the method of Gibbs et al. to predetermine the optimal path leading to an ROI [8]. Later, during live bronchoscopy, a sensor continuously measures the insertion depth and roll angle of the real bronchoscope. In real time, the prediction engine then compares the observed insertion depth from the sensor to the precomputed insertion depths of each view site along the predefined path. The prediction engine selects the predicted bronchoscope location as the view site having a precomputed insertion depth that is closest to the observed insertion depth. We use the observed rotation measurement (roll angle) to rotate the default viewing direction at the selected view site. The location and view direction then help generate an endoluminal rendering that provides simple navigational instructions.

### Measurement Sensor

All virtual-endoscopy-driven IGI systems require a fundamental connection between the virtual space and physical space. In ENB-based systems, the connection involves a registration of the EM field in physical space to the 3D MDCT data representing virtual space. Image-based bronchoscopy systems draw upon some form of registration between the live bronchoscopic video of physical space and VB renderings devised from 3D MDCT-based virtual space. Our method uses a fundamentally different connection. Live measurements of the bronchoscope's movements through physical space, as made by a calibrated sensor mounted outside a patient's body, are linked to the virtual-space representation of the airway tree N.

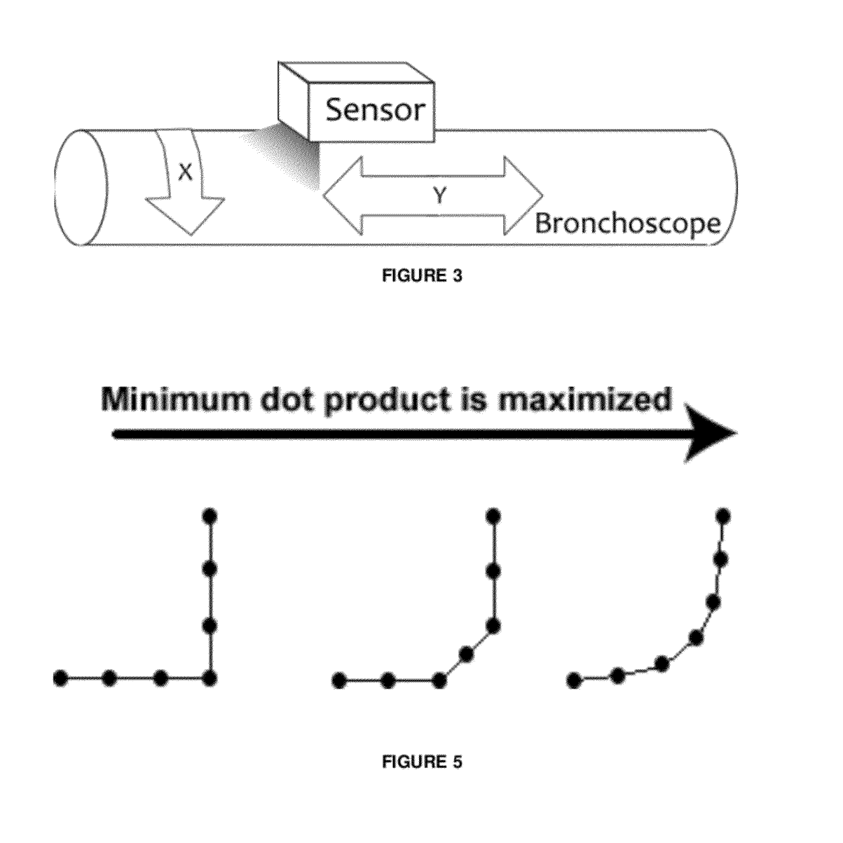

The sensor tracks the bronchoscope surface that moves past the sensor. If the sensor is oriented correctly, the “Y” component (up-down) gives the insertion depth, while the “X” component (left-right) gives the roll angle (FIG. 3). Any device that provides insertion and rotation measurements could be used. Examples of such devices include optical sensors similar to those found in optical computer mice or tactile rotary encoders. The system explained by Eickhoff et al. uses an external position sensor to measure a colonoscope's insertion depth for use in a computer-articulated-colonoscope system [7]. We use a similar sensor in our system that also records rotation information.

Because a bronchoscope is a torsionally-stiff, semi-rigid object, any roll measured along the shaft of the bronchoscope will propagate throughout the entire shaft [21]. Simply stated, if the physician rotates the bronchoscope at the handle, the tip of the bronchoscope will also rotate the same amount. This is what gives the physician control to maneuver the bronchoscope.

### The Prediction Engine and Bronchoscope Models

The measurement sensor sends the insertion depth and roll angle measurements to a prediction engine running in real time on a computer. An algorithm uses these measurements to predict a view site location and orientation. We now discuss bronchoscope models and how they can be used for calculating insertion depths to view sites.

Previous research by Kukuk et al. focused on modeling bronchoscopes to gain insertion-depth estimates for robotic planning [21, 23, 18, 22, 20, 19]. Kukuk's goal was to preplan a series of bronchoscope insertions, rotations, and tip articulations to reach a target. In doing so, the method calculates an insertion depth to points in an airway tree using a search algorithm. It models a bronchoscope as a series of rigid “tubes” connected by “joints.” A bronchoscope's shape is determined by the lengths and diameters of the tubes as well as how the tubes connect to each other. Each joint allows only a discrete set of possible angles between two consecutive tubes. Using a discrete set of possible angles reduces the search space to a finite number of solutions. However, the solution space grows exponentially as the number of tubes increases. In practice, the human airway-tree structure reduces the search space, and the algorithm can find solutions in a feasible time. However, the method cannot find a solution to any arbitrary location in the airways in a feasible time. Therefore, we use a different method for calculating a bronchoscope model, as explained next.

Similar to the method of Kukuk et al., our bronchoscope-model calculation is done offline to allow for real-time bronchoscope location prediction. The purpose of a bronchoscope model is to precompute and store insertion depths to every airway-tree view site so that later, during bronchoscopy, they may be compared to true insertion measurements provided by the sensor. Precomputation allows for an inverse lookup of the predicted location during a live bronchoscopy.

To begin our description of the bronchoscope model, consider an ordered list of 3D points {uₐ, u_{b}, . . . , uₖ}, where each uₐ, u_{b}, etc.εV_{seg}, uₐ is the proximal end of the trachea, and uₖ is a view site. Connecting each consecutive pair of 3D points creates a list of connected line segments that define our bronchoscope model S(k), as shown below:

S(k)={ u̅ₐ̅u̅_̅{̅b̅}̅, u̅_̅{̅b̅}̅u̅_̅{̅c̅}̅, . . . , u̅ᵢ̅u̅ⱼ̅, u̅ⱼ̅u̅ₖ̅}. (2)

This representation of a bronchoscope approximates the bronchoscope shape when the bronchoscope tip is located at view site k. By converting each line segment u̅_̅{̅f̅}̅u̅_̅{̅g̅}̅ into a vector û_{f}, we can sum the magnitude of all vectors to calculate the insertion depth I_{d}(k) to view site k using the equation below:

$${I}_{d} ( k ) = {\sum }_{x = a}^{k - 1} {‖ {\hat{u}}_{x} ‖}_{2} , \qquad ( 3 )$$

where x iterates through the list of ordered vectors and is the ∥ûₓ∥₂ is the L²-norm of vector ûₓ. Using this method, we can calculate a separate insertion depth to each view site along the centerlines of all airway-tree branches.

Unlike the method of Kukuk, which uses 3D tubes connected by joints, we approximate a bronchoscope as a series of line segments that have diameter 0; i.e., S(k) technically models only the central axis of the real bronchoscope [21]. As this approximation unrealistically allows the bronchoscope model to touch the airway wall in the segmentation V_{seg}, we prefer to account for the non-zero diameter of the real bronchoscope in our bronchoscope-model calculation.

To do this, we first point out that the central axis of the real bronchoscope can only be as close as its radius r to the airway wall. To account for this, we erode the segmentation of N, V_{seg}, using the following equation:

\hat{V}_{seg}=V_{seg} [symbol] b, (4)

where b is a spherical structuring element having a radius r and [symbol] is the morphological erosion operation. In the eroded image \hat{V}_{seg}, if the bronchoscope model touches the airway wall, then the central axis of the bronchoscope is a distance r from the true airway wall.

\hat{V}_{seg} loses small branches that have a diameter <2r. Because we do not want to exclude any potentially plausible bronchoscope maneuvers, we force the centerlines of small branches to be contained in \hat{V}_{seg} as well as all voxels along the line segments between any two consecutive view sites. Overriding the erosion ensures that we can calculate a bronchoscope model for every view site. Thus, \hat{V}_{seg} is redefined to only include the voxels that remain after the erosion and view-site inclusion.

As discussed below, we consider three methods for creating a bronchoscope model: (a) Centerline; (b) Dijkstra-based; and (c) Dynamic Programming.

### Centerline Model

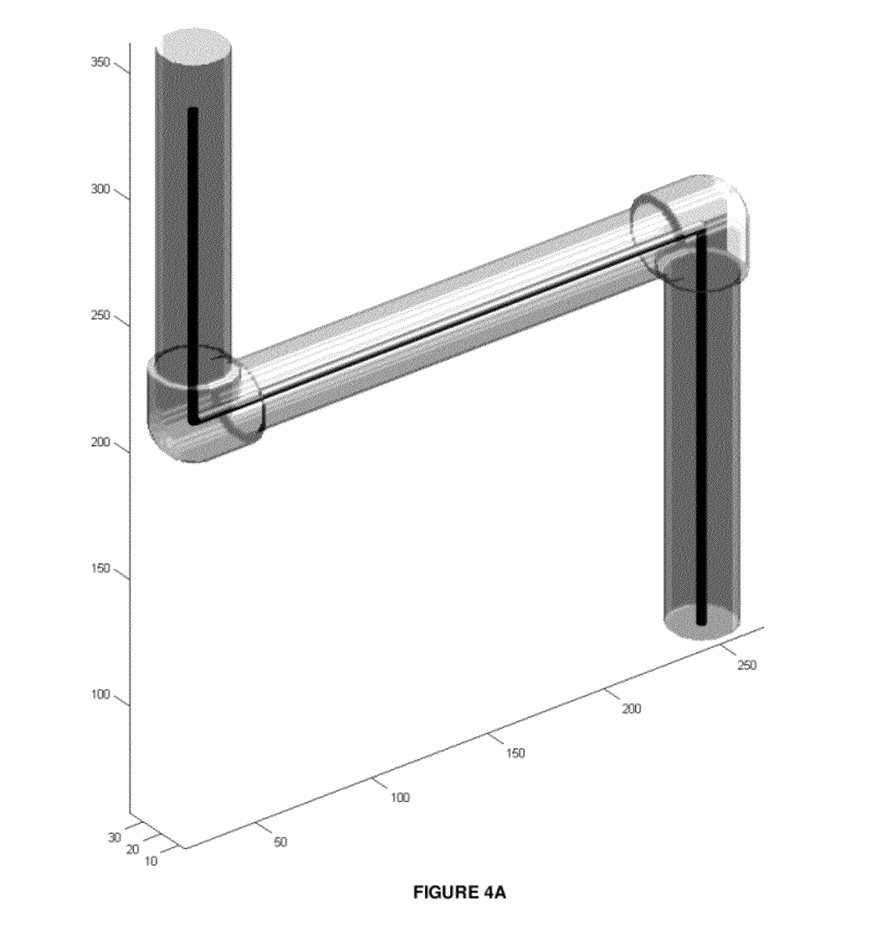

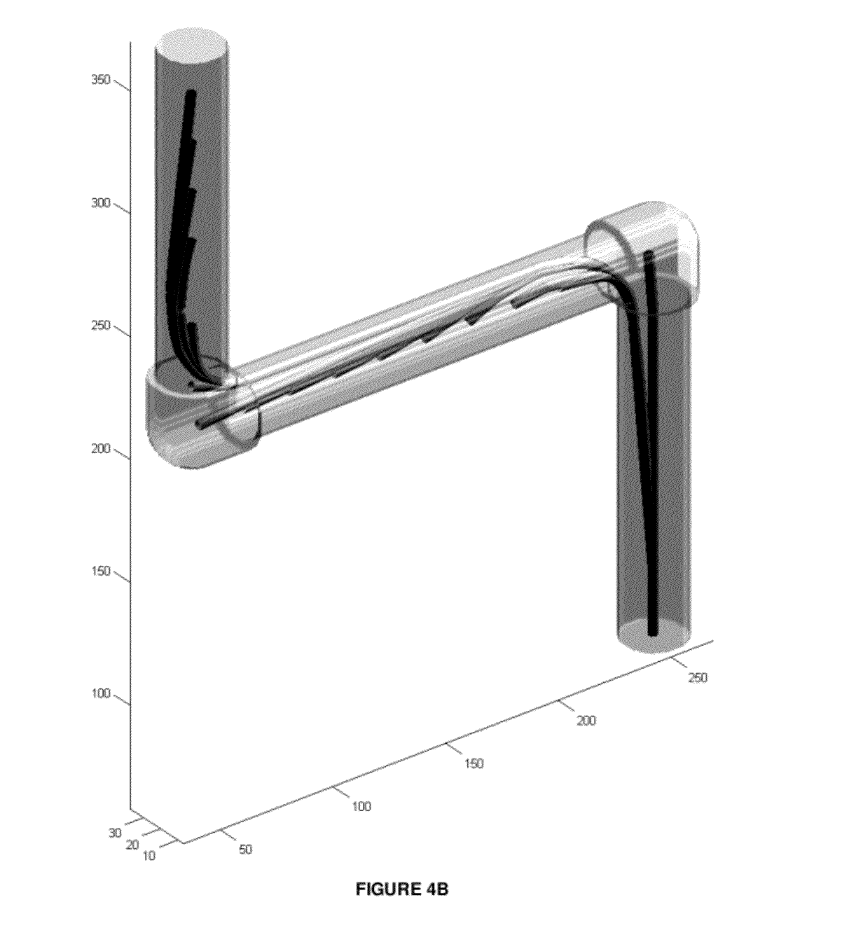

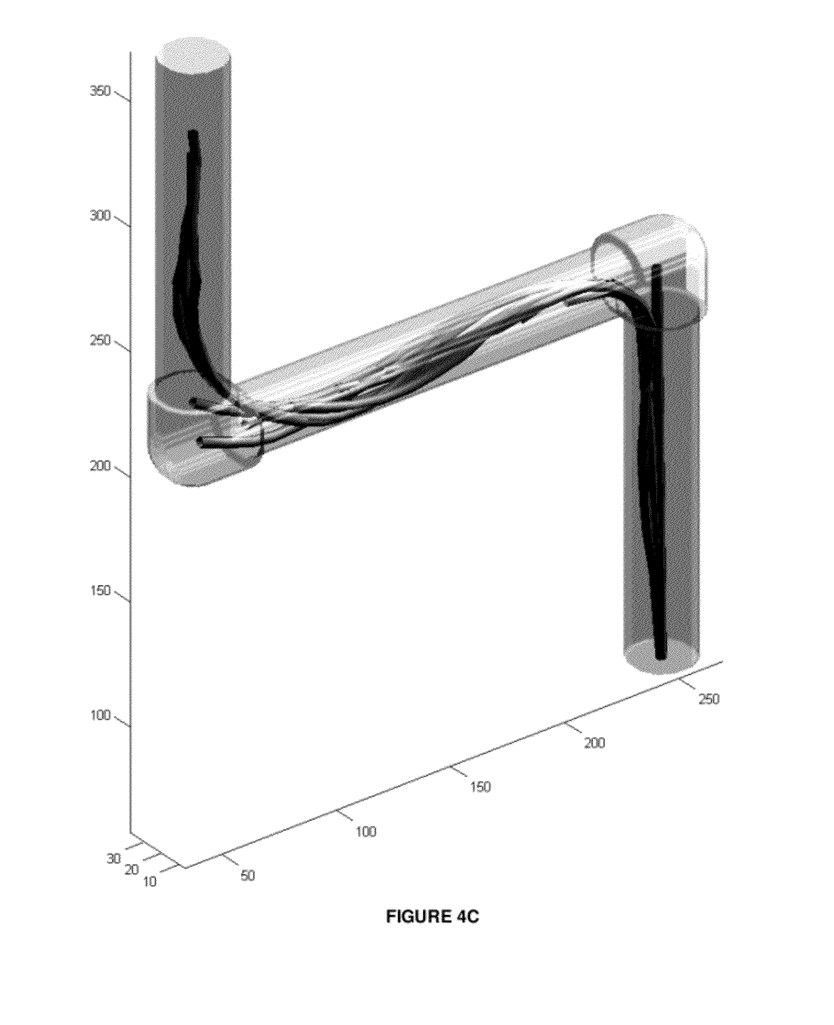

The centerline model is the simplest bronchoscope model. The list of 3D points S(k), terminating at an arbitrary view site k, consists of all ancestor view sites traced back to the proximal end of the trachea. This method gives a rough approximation to a true bronchoscope, because the view sites never touch the walls of the segmentation, which is not the case with a real bronchoscope in N. Furthermore, a real bronchoscope does not bend around corners in the same manner as the centerlines can. FIG. 4A depicts an example centerline model in a rendered PVC pipe.

### Dijkstra-based Model

Dijkstra's shortest-path algorithm finds the shortest distance between two nodes in an arbitrary graph, where the distance depends on edge weights between nodes [17]. For computing a bronchoscope model, we use Dijkstra's algorithm as follows. First, the edge weight between two nodes, j and k, is defined as:

w(j,k)=w_{E}(j,k)+wₐ(j,k), (5)

where j and k are voxels in \hat{V}_{seg}, w_{E}(j,k) is the Euclidean distance between j and k, and wₐ(j,k) is the edge weight due to the angle between the incident vectors coming into voxels j and k. w_{E}(j,k) is given by:

$${w}_{E} ( j , k ) = \sqrt{{\sum }_{d = 1}^{3} {( {k}_{d} - {j}_{d} )}^{2}} , \qquad ( 6 )$$

where k_{d} is the d^{th} coordinate of the 3D point k. wₐ(j,k) is given by:

wₐ(j,k)= [symbol] β [symbol] 1−(ĵᵢ·\hat{k}ᵢ) [symbol] [symbol] ^{p}, (7)

where ĵ, is the normalized incident vector coming in to voxel j, \hat{k}ᵢ is the normalized incident vector coming in to voxel k from j, (m·n) represents the dot product of vectors, in and n, and β and p are constants.

These two weight terms serve different purposes. In the cost (5), w_{E}(j,k) penalizes longer solutions, while wₐ(j,k) penalizes solutions where the bronchoscope model makes a sharp bend. This encourages solutions that put less stress on the bronchoscope.

The incident vectors, ĵᵢ and \hat{k}ᵢ in (7), are known during model computation, as Dijkstra's algorithm is greedy [17]. It greedily adds nodes to a set of confirmed nodes with known shortest distances. In our implementation, j is already in the set of known shortest-distance nodes.

Algorithms 1 and 2 detail our implementation of the Dijkstra-based bronchoscope model. Algorithm 1 computes a bronchoscope model for each view site in an airway tree and stores them in a data structure. Algorithm 2 extracts the bronchoscope model to a view site vₛ out of the data structure from Algorithm 1. FIG. 4B depicts Dijkstra-based example bronchoscope models for the PVC pipe.

Algorithm 1 Dijkstra-based bronchoscope-model generation algorithm.

| | |
|---|---|
| Input: | |
| V [symbol] cg | /* Segmentation */ |
| r | /* Root site in proximal end of trachea */ |
| Data Structures: | |
| MinDist[x] | /* Minimum distance to each segmentation voxel x */ |
| Confirmed[x] | /* Boolean array indicating if x has been processed */ |
| Q | /* Priority Queue of voxels sorted by distance */ |
| Output | |
| PreviousNode[x] | /* Array indicating x's parent voxel */ |
| Algorithm: | |
| 1. for all x ε V [symbol] cg do | /* Initialize data structures */ |
| 2. MinDist[x] ← ∝; | |
| 3. previousNode[x] ← r; | |
| 4. Confirmed[x] ← false; | |
| 5. Q.push(r); | /* Insert voxel r onto priority queue Q */ |
| 6. MinDist[r] ← 0; | /* Initialize minimum distance to r */ |
| 7. while Q.size > 0 do | /* Iterate while there are still voxels to process */ |
| 8. C ← Q.top; | /* Retrieve voxel with shortest distance */ |
| 9. Q.pop; | /* Remove C from Q */ |
| 10. if Confirmed[C] = false | /* Ensure we haven't processed voxel C already */ |
| 11. Confirmed[C] ← true; | /* Mark voxel C as processed */ |
| 12. for all voxels u εNeigh(C) | /* Iterate through neighbors of C */ |
| 13. if Confirmed[u] = false | /* Ensure u has not been processed */ |
| 14. if Dist(C, u)+ MinDist[C] <MinDist[u] then | |
| 15. MinDist[u] ← Dist(C, u)+ MinDist[C]; | /* u now has a lower cost with C as its parent */ |
| 16. Q.push(u); | /* Put u on priority queue with new distance */ |
| 17. PreviousNode[u] ← C; | /* Update u's parent */ |
| 18. Output PreviousNode; | /* Output PreriousNode array for later processing */ |

[symbol]

indicates data missing or illegible when filed

Algorithm 2 Dijkstra-based backtracking algorithm producing a
bronchoscope model leading to view site uₛ.

| | |
|---|---|
| Input: | |
| PreviousNode[x] | /* Array indicating x's parent voxel */ |
| r | /* Root site in proximal end of trachea */ |
| uₛ | /* Terminating view site of desired |
| | bronchoscope model */ |
| Output | |
| S(uₛ) | /* Bronchoscope model defined by (2) */ |
| Algorithm: | |
| 1. z ← uₛ; | /* Initialize data structures */ |
| 2. S.push_back(z); | /* Fill list S with 3D points by back |
| | tracking */ |
| 3. while z ≠ r do | |
| 4. z ← PreviousNode[z]; | |
| 5. S.push_back(z); | |
| 6. Output S(uₛ) | /* Output bronchoscope model to uₛ */ |

Because we are selecting discrete points to be members of the set of bronchoscope-model points, we have no guarantee that the line segment connecting these two points will remain in the segmentation at all times. The “Dist” function in Algorithm 1 checks if a line segment between two model points exits the segmentation, by stepping along the line segment at a small step size and ensuring that the nearest voxel to each step point is inside the segmentation.

### Dynamic Programming Model

Dynamic programming (DP) algorithms find optimal solutions based on an optimization function for problems that have optimizable overlapping subproblems [17]. Before defining our use of DP for defining a bronchoscope model, it is necessary to recast the bronchoscope-model problem. Recall that S(k) is a list of connected line segments per (2). Similar to (3), we again represent a line segment as a vector. However, this time we represent the line segment using the end point of the line segment. Therefore, line segment u̅ⱼ̅u̅ₖ̅ is denoted as vector \hat{k}ᵢ that starts at uⱼ and points to uₖ. Vector \hat{k}ᵢ represents the incident vector coming into voxel k. Using this definition, it is possible to find the solution that terminates at a point where the lowest dot product among all consecutive normalized vectors in one bronchoscope model is maximized. This is akin to finding the solution that minimizes sharp bends. FIG. 5 depicts a toy example illustrating this optimization process. The optimal bronchoscope model S(k,l) to a voxel k using l line segments (or links) is calculated using:

$$S ( k , l ) = {max}_{t ∈ N ( k )} ( min ( S ( t , l - 1 ) , ( {\hat{k}}_{t} \cdot {\hat{t}}_{i} ) ) ) , \qquad ( 8 )$$

where N (k) is a neighborhood about voxel k, \hat{k}ᵢ is the normalized vector from t to k, and \hat{t}ᵢ is the incident vector coming into voxel t from its parent voxel.

Using this method, we calculate an optimal bronchoscope model from the root site to every voxel in \hat{V}_{seg}. In the memorized DP framework, solutions are built from the “bottom up,” and results are saved so later recalculation is not needed [17]. First, the DP algorithm determines the optimal solution to every voxel using only one link and an automatically generated unit vector coming into the root site \hat{r}ᵢ. The solution to an arbitrary voxel xε\hat{V}_{seg} is simply the line segment from the root site to x. The algorithm stores the dot product between \hat{r}ᵢ and the normalized vector from the root site to x in a 2D array that is indexed by x and the number of links used.

Next, the algorithm determines the optimal solution to every voxel using two links. To find the optimal solution using two links, the method uses the previously calculated data from the optimal solution with one link. The algorithm calculates the solution to an arbitrary voxel using two links by adding a link from each neighbor to x, providing several candidate bronchoscope models to voxel x. For each candidate bronchoscope model, the method next calculates the minimum dot product found for the solution with one link (from the 2D array) and the new dot product (created with the addition of the new link). Finally, the method chooses the bronchoscope model with the maximum of all the minimum dot products. This is akin to selecting the bronchoscope model whose sharpest angle is as straight as possible, given the segmentation. The same procedure is carried out for all other voxels. We store the maximum of the minimum values in the 2D array saving the best solution to each voxel. Solutions are built up to a user-defined number of links in this manner. The algorithm also maintains another 2D table that contains back pointers. This table indicates the parent of each voxel so that we can retrieve the voxels belonging to S(k).

Algorithm 3 specifies the DP algorithm for computing all of the bronchoscope models for a given airway tree segmentation. Algorithm 4 shows how to trace backwards through the output of Algorithm 3 to retrieve a bronchoscope model leading to view site vₛ. FIG. 4C depicts the DP model for the PVC pipe.

Algorithm 3 DP bronchoscope-model generation algorithm.

| | |
|---|---|
| Input: | |
| V [symbol] cg | /* Segmentation */ |
| r | /* Root site in proximal end of trachen */ |
| links | /* Maximum number of line segments */ |
| Data Structures: | |
| LowDotProd[x,l] | /* The optimal solution to voxel x using */ |
| Output | /* l line segments */ |
| BackPtr[x,l] | /* Array indicating x's parent voxels in */ |
| Algorithm: | /* bronchoscope model with l line segments */ |
| 1. for all x ε V [symbol] cg do | /* Initialize data structures */ |
| 2. for 1 ← 0....,links − 1 do | |
| 3. if l = 0 then | |
| 4. LowDotProd[x,0] ← DotProd(n₁, normalize(x − 0)); | |
| 5. else | |
| 6. LowDotProd[x,l] ← x; | |
| 7. BackPtr[x,l] ← r; | |
| 8. for 1 ← 0....,links − 1 do | |
| 9. for all x ε l, do | |
| 10. LowDotProd[x,l] ← LowDotProd[x,l − 1]; | /* lacumbent optimal solution is from solution */ |
| 11. BackPtr[x,l] ← BackPtr[x,l − 1]; | */ with l − 1 links */ |
| 12. for all voxels n εNeigh(C) do | |
| 13. if min(DotProd(x₁,n₂),LowDotProd[n,l − 1]) > LowDotProd[x,l] then | |
| 14. LowDotProd[x,l] ← min(DotProd(x₁, n₁).LowDotProd[n,l − 1]); | /* Found new optional route */ |
| 15. BackPtr[x,l] ← n; | |
| 16. Output BackPtr; | /* Output BackPtr array for later processing */ |

[symbol]

indicates data missing or illegible when filed

Algorithm 4 DP backtracking algorithm producing a bronchoscope
model leading to view site uₛ.

| | |
|---|---|
| Input: | |
| BackPtr[x,l] | /* Array indicating x's parent voxel in solution |
| | with l line segments*/ |
| r | /* Root site in proximal end of trachen */ |
| links | /* Maximum number of allowable links */ |
| r [symbol] | /* Terminating view site of desired |
| | bronchoscope model */ |
| Output | |
| S(u_{A}) | /* Bronchoscope model defined by (2) */ |
| Algorithm: | |
| 1. [symbol] ← u [symbol] ; | /* Initialize data structures */ |
| 2. S.push_back(x); | /* Fill list S with 3D points by back tracking */ |
| 3. l ← links − 1; | |
| 4. while x ≠ r do | |
| 5. x ← BackPtr[x,l]; | |
| 5. S.push_back(x); | |
| 5. 1 ← l − 1; | |
| 6. Output S(uₛ); | /* Output bronchoscope model to u [symbol] */ |

[symbol]

indicates data missing or illegible when filed

### Implementation

We implemented the bronchoscope tracking method for testing purposes. The computer-based prediction engine and the bronchoscope model generation software were written in Visual C++. with MFC interface controls. We interfaced two computer mice to the computer. The first served as a standard computer mouse to interface to software. The second mouse was a Logitech MX 1100 wireless laser mouse that served as the measurement sensor. The measurement-sensor inputs were tagged as such so that its input could be identified separately from the standard computer-mouse inputs. The method ran on a computer with two 2.99 GHz processors and 16 GB of RAM for both the precomputation of the bronchoscope models and for later real-time bronchoscope tracking. During tracking, every time the sensor provided a measurement, the tracking method invoked the prediction engine to predict a bronchoscope location using the most recent measurements.

### Results

We performed two tests. The first used a PVC-pipe setup to compare the accuracy of the three bronchoscope models for predicting a bronchoscope location, while the second test involved a human airway-tree phantom to test the entire real-time implementation. For both experiments, the Dijkstra-based model parameters were set as follows: β=100, p=3.5, neighborhood=25×25×25 cube (±12 voxels in all three dimensions). The DP model parameters were set as follows: neighborhood=25×25×25 cube, max number of line segments=60. Note that the optimal solutions for all view sites considered in our tests required fewer than the maximum allowed 60 line segments.

### PVC-pipe Experiment

The PVC-pipe setup involved three PVC-pipe segments connected with two 90″ bends along with 26 screws inserted through the side of the complete PVC pipe (FIG. 4). The screws served as navigational targets allowing for 25 targets with an insertion depth of up to 480 mm (screw spacing=2 cm). When the screws were inserted to a specified depth, the tips of the screws touched the central axis of the PVC-pipe assembly. Because we knew the geometry of the physical PVC pipe, we were able to create a virtual version, allowing for straightforward computer-based calculation of the bronchoscope models. Each screw location was also known in the virtual model.

Given this setup, the bronchoscope could be inserted to each screw location to compare a predicted bronchoscope tip location to the real known bronchoscope tip location. The test ran as follows:

1. Insert the bronchoscope into the PVC pipe to the first screw tip (location serves as a registration location), using the bronchoscopic video feed for guidance and verification.

2. Place tape around the bronchoscope shaft to mark the insertion depth to the first screw location.

3. Advance the bronchoscope to the next screw tip, as in step 1.

4. Place tape around the bronchoscope shaft to mark the insertion depth to the current screw tip location.

5. Repeat steps 3 and 4 until the last screw tip location is reached.

6. Remove the bronchoscope and manually measure the distance from the first tape mark to all other tape marks, providing a relative insertion depth to each screw tip location.

7. Run the prediction algorithm using manually measured insertion depths relative to the first screw for each of the three bronchoscope models.

8. Compute the Euclidean distance between the predicted locations and the actual screw tip location.

We repeated this test over three trials and averaged the results of the three trials (Table I). The centerline model performed the worst, while the DP model performed the best. On average, the DP model was off by <2 mm. The largest error occurred in PVC-pipe locations where we utilized the bronchoscope's articulating tip to get the bronchoscope to touch a screw; we detected an error of −19 mm to the screw located just beyond the second 90% bend. Once we advanced the bronchoscope 2 cm beyond that location to where the articulating tip was not heavily utilized, the error shrank to −3 mm.

TABLE I
Euclidean distance errors (mm) of predicted locations
and actual locations over three trials for the PVC model.

| | CM | DM | DP |
|---|---|---|---|
| Average | −13.0 ± 11.6 | 3.2 ± 6.4 | 1.8 ± 6.3 |
| Median | −12.5 | 1.7 | 1.2 |
| Range | −49-1.9 | −17.5-19.5 | −17.0-19.5 |

A negative value indicates that the predicted location is not as far into the PVC model as the actual location.
CM = Centerline Model,
DM = Dijkstra-based Model,
DP = Dynamic-Programming Model.

### Phantom Experiment

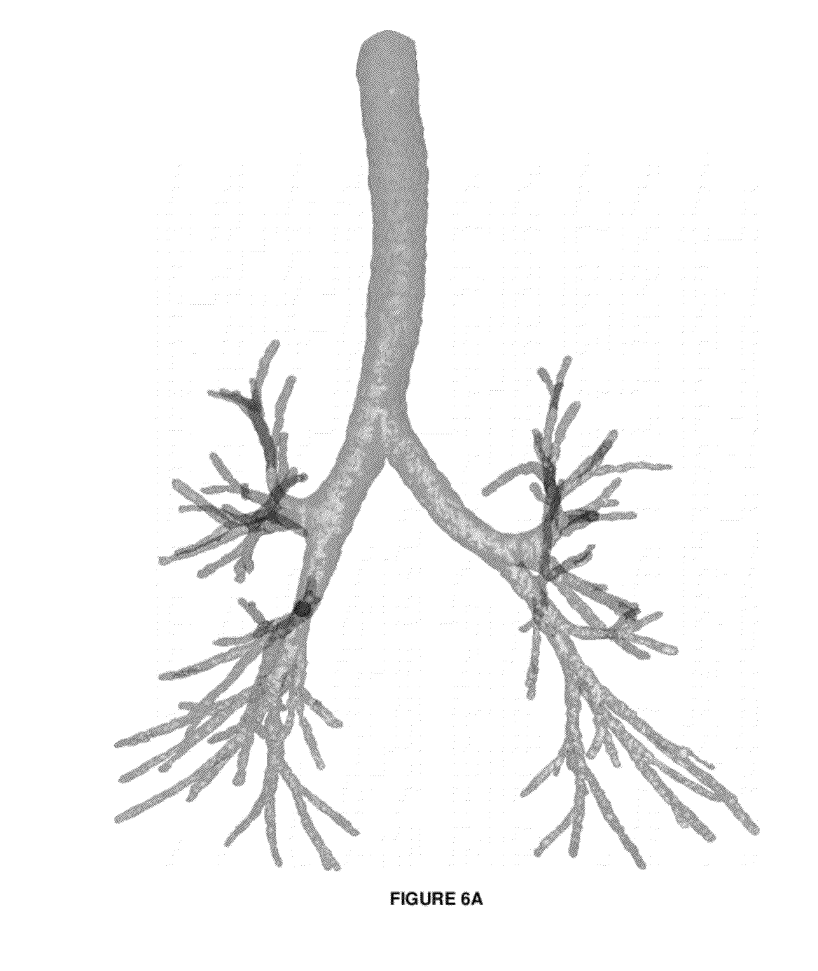

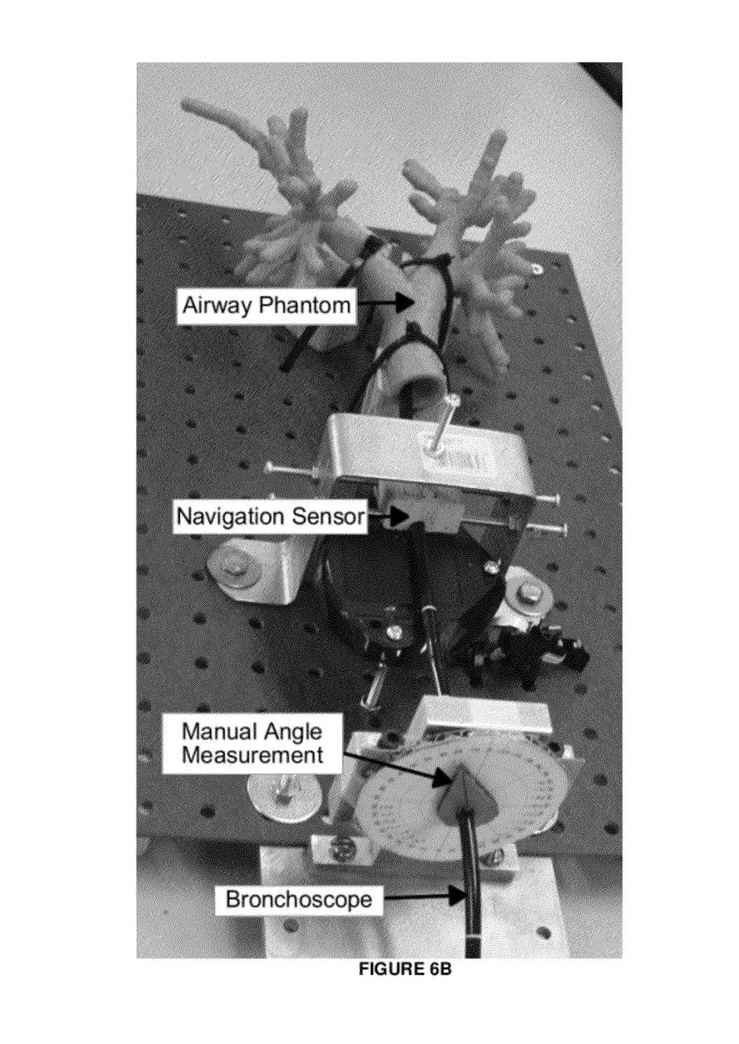

The second experiment evaluated the entire implementation. During this experiment, we maneuvered a bronchoscope through an airway tree phantom. A third party constructed the phantom using airway-surface data we extracted from an MDCT scan (case 21405-3a). Thus, the phantom serves as the real physical space, while the MDCT scan serves as the virtual space. The experimental apparatus (FIG. 6B) allows us to record two sets of insertion and rotation measurements: 1) real-time sensor measurements; 2) true hand-made measurements. We used the measurement-sensor mouse discussed herein to provide the real-time sensor measurements. The hand-made measurements were recorded manually using tape and a mounted angle scale (FIG. 6B). Before the experiment, we placed tape around the bronchoscope at 3 mm increments to attain 25 discrete insertion depths. Inserting the bronchoscope to each insertion depth provided a real bronchoscopic video frame. At each of the 25 discrete insertion depths, we determined a ground-truth 3D location by maneuvering a virtual camera through a virtual airway tree derived from the MDCT data to manually align the VB view to the bronchoscopic video frame. It is worth reiterating that the method is for continuous tracking, but to analyze how well it continually tracks the bronchoscope, we recorded ground-truth measurements at discrete locations.

Prior to the test, the bronchoscope shaft was covered with semi-transparent tape to allow for the optical sensor to have a less reflective surface to track. During the test, we inserted the bronchoscope to each tape mark, following a 75 mm preplanned route to a fictional ROI, depicted in FIG. 6A, while the system continuously tracked position in real-time without technician assistance. The steps of the experiment are listed below:

1. Insert the bronchoscope to the first tape mark to register the virtual space and the physical space. Record the roll angle by using the manual angle measurement apparatus (FIG. 6B).

2. Insert the bronchoscope to the next tape mark.

3. Record the three different bronchoscope predictions produced by the three different bronchoscope models.

4. Record the true insertion depth (known by multiplying the tape mark number by 3 mm) and the true roll angle of the bronchoscope (recorded from apparatus).

5. Remove the bronchoscope.

6. Repeat steps 1 through 5 inserting to each subsequent tape mark in step 2 until the target is reached.

We calculated errors using both the hand-made measurements (representing an error-free sensor) and the sensor measurements, providing four different sets of measurements. Error I_{H} is the Euclidean distance between the predicted and true bronchoscope locations using the hand-made measurements. Error II_{H} is the Euclidean distance between the predicted bronchoscope location and closest view site to the true bronchoscope location using hand-made measurements. Error II_{H} does not penalize our method for constraining the predicted location to the centerlines. These errors quantify the error using a hypothetical, error-free sensor and therefore quantify the error in a system with a perfect sensor. The next two errors, I_{S} and II_{S}, use the measurements provided by the sensor instead of the hand-made measurements, providing the overall error of the method. Table II shows error I_{H} and II_{H}, while Table III shows error I_{S} and II_{S} evaluating the whole method.

TABLE II
Phantom experiment Euclidean distance error (mm) between true and predicted
bronchoscope locations using hand-made measurements.

| | Error From True Location (mm) - I_{H} | | | Error along centerline (mm) - II_{H} | | |
|---|---|---|---|---|---|---|
| | CM | DM | DP | CM | DM | DP |
| Average | −2.9 ± 3.7 | 3.4 ± 3.5 | 1.3 ± 4.6 | −1.3 ± 1.0 | 0.8 ± 1.0 | 0.1 ± 0.9 |
| Median | −4.4 | 4.7 | 3.2 | −1.1 | 0.8 | 0 |
| Range | −6.2 to 5.3 | −5.6 to 6.6 | −6.2 to 6.3 | −3.4 to 0.4 | −0.9 to 3.3 | −2.0 to 2.2 |

A negative value indicates that the predicted location is not as far into the phantom as the actual location.

TABLE III
Phantom experiment Euclidean distance error (mm) between true and predicted
bronchoscope locations using measurements provided by an optical sensor.

| | Error From True Location (mm) - I_{S} | | | Error along centerline (mm) - II_{S} | | |
|---|---|---|---|---|---|---|
| | CM | DM | DP | CM | DM | DP |
| Average | −3.6 ± 3.3 | 3.0 ± 4.0 | 1.7 ± 4.6 | −1.4 ± 1.7 | 0.7 ± 1.9 | −0.02 ± 1.7 |
| Median | −4.8 | 4.3 | 3.3 | −1.1 | 1.3 | 0 |
| Range | −7.3 to 5.0 | −6.6 to 6.8 | −6.7 to 6.5 | −6.8 to 0 | −5.4 to 2.9 | −5.7 to 2.0 |

A negative value indicates that the predicted location is not as far into the phantom as the actual location.

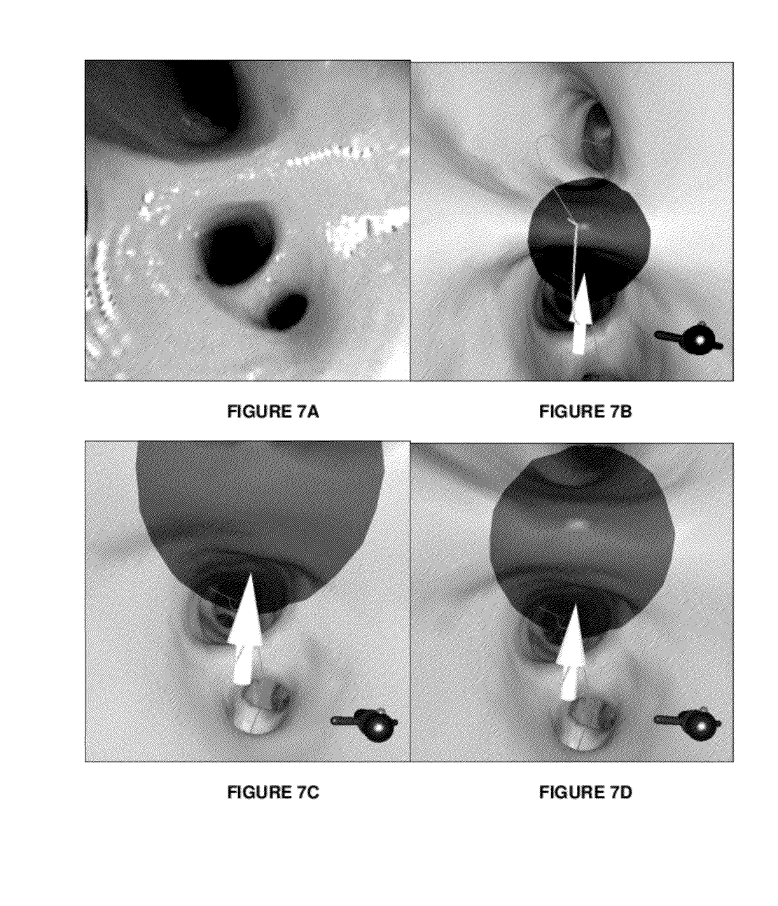

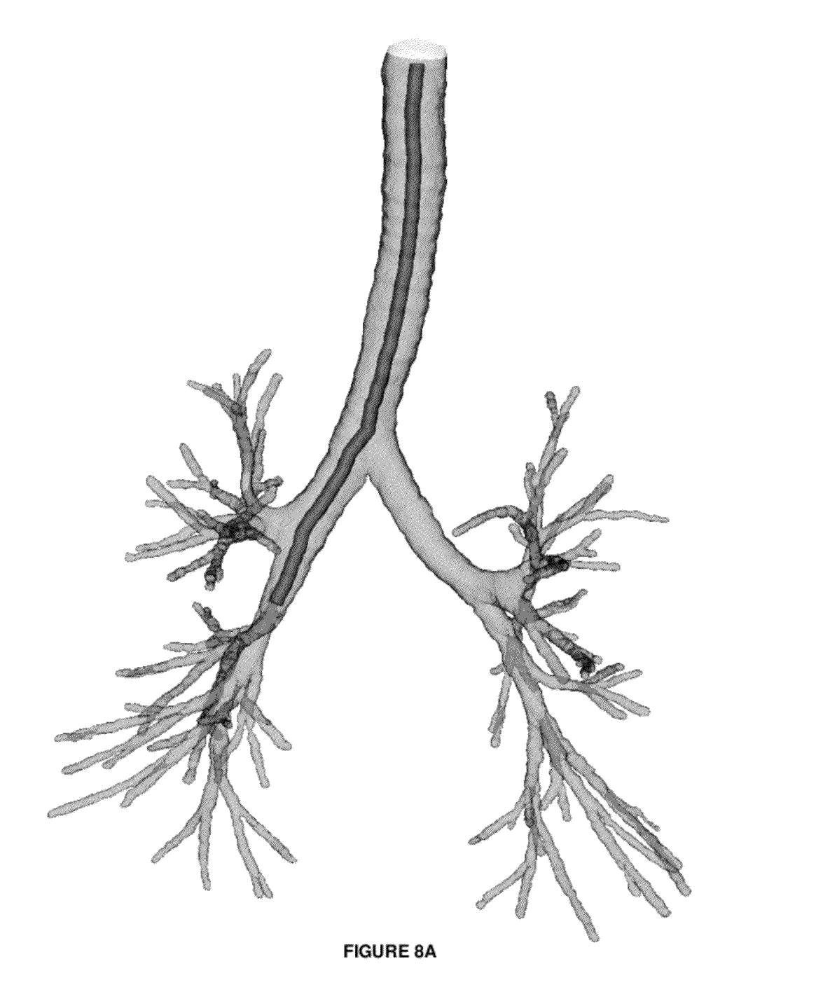

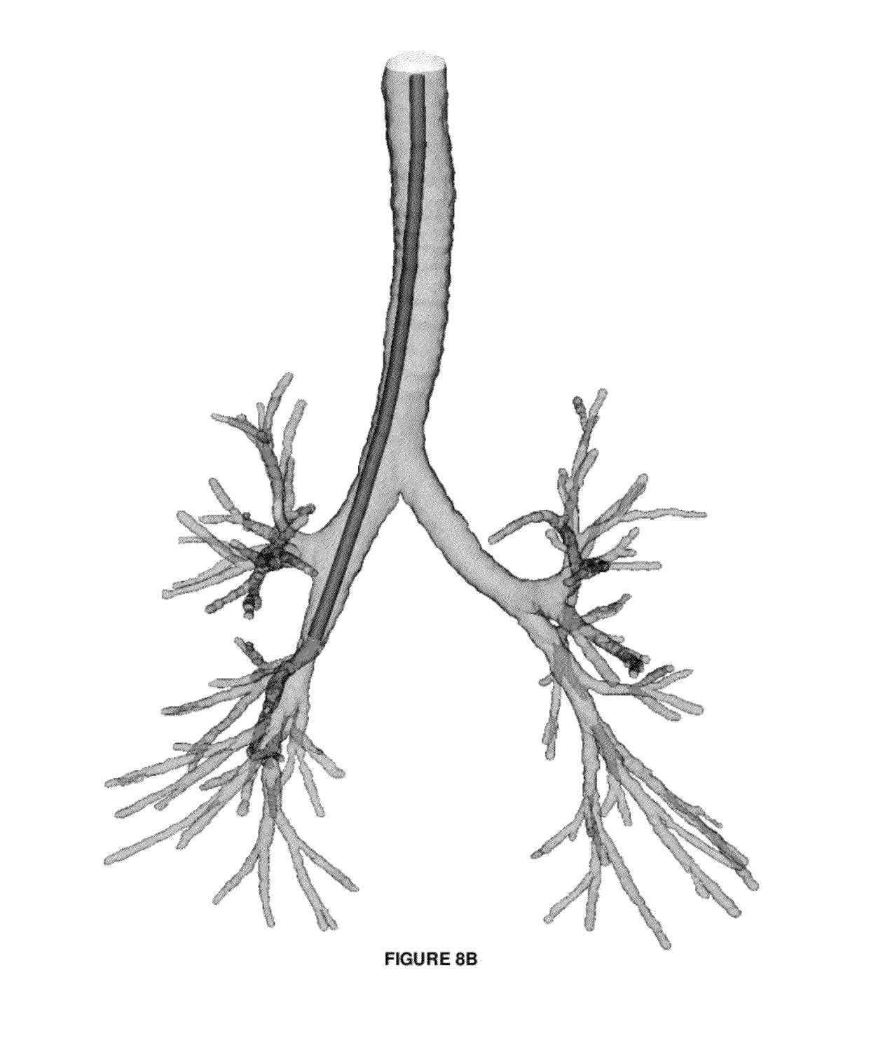

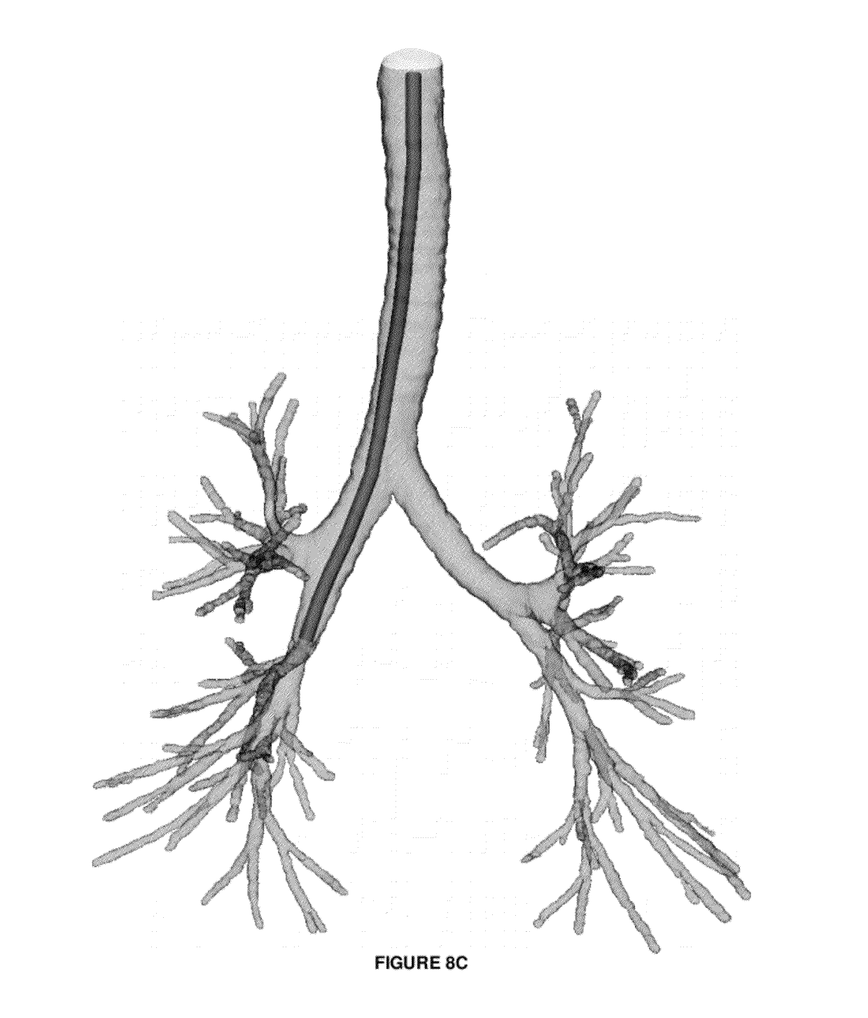

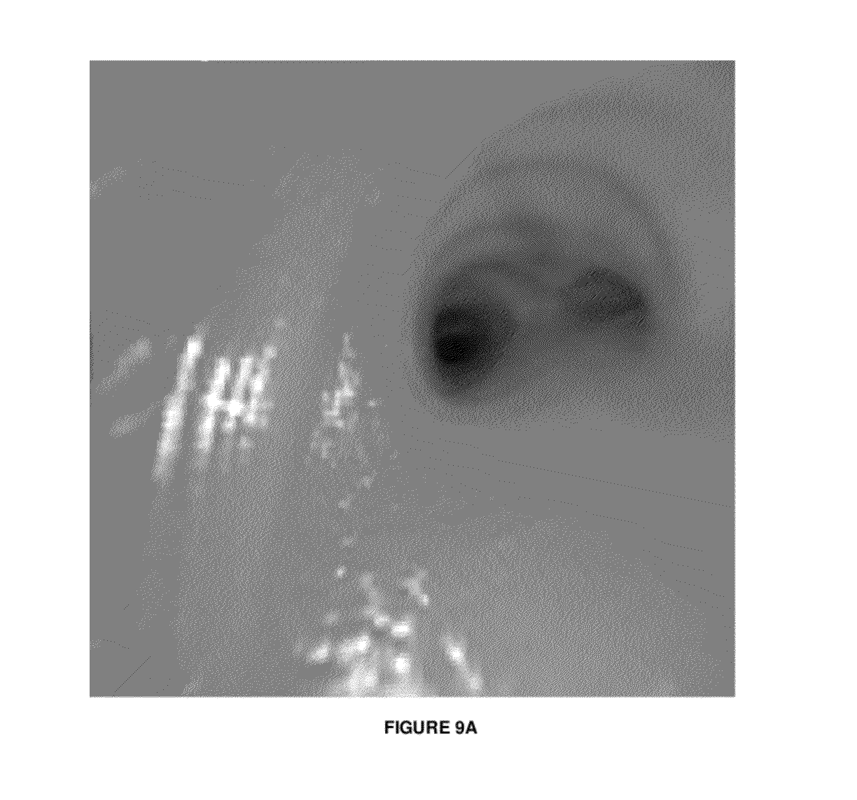

Recording both hand-made measurements and the optical sensor measurements allowed us to determine how accurate the mouse sensor was. Table IV quantifies how far off the mouse sensor measurements were from the hand-made measurements during the phantom experiment. FIGS. 7A-7D shows three different predicted views from the three bronchoscope models using the sensor measurements next to the live video frame near the ROI. FIGS. 8A-8C show the bronchoscope models corresponding to the views in FIGS. 7A-7D.

TABLE IV
Error from the mouse sensor compared to hand-made measurements.

| | Insertion Depth Error | Roll Angle Error |
|---|---|---|
| | (mm) | (deg) |
| Average | −0.2 ± 1.6 | 10.8 ± 11.1 |
| Median | 0.1 | 5.7 |

### Discussion

The centerline model consistently overestimated the bronchoscopic insertion depth required to reach each view site. The Dijkstra-based model on average underestimated the required insertion depth. The insertion depth calculated from the DP solution tends to be between the other two models, indicating that it might be the best bronchoscope model for estimating an insertion depth to a location in the lungs among the three tested.

Tables II and III indicate that the accuracy of the bronchoscope location prediction using the DP model is within 2 mm of the true location on average. Given that an ROI has a typical size of roughly 10 mm or greater in diameter, an average error of only 2 mm in accuracy is acceptable for guiding a physician to ROIs. Furthermore, a typical airway branch is anywhere between 8 mm and 60 mm in length. In lower generations (close to trachea) the branch lengths tend to be longer, and in higher generations (periphery) they tend to be shorter. Thus, in airway branches, an error of only 2 mm is acceptable to prevent misleading views from incorrectly guiding a physician.

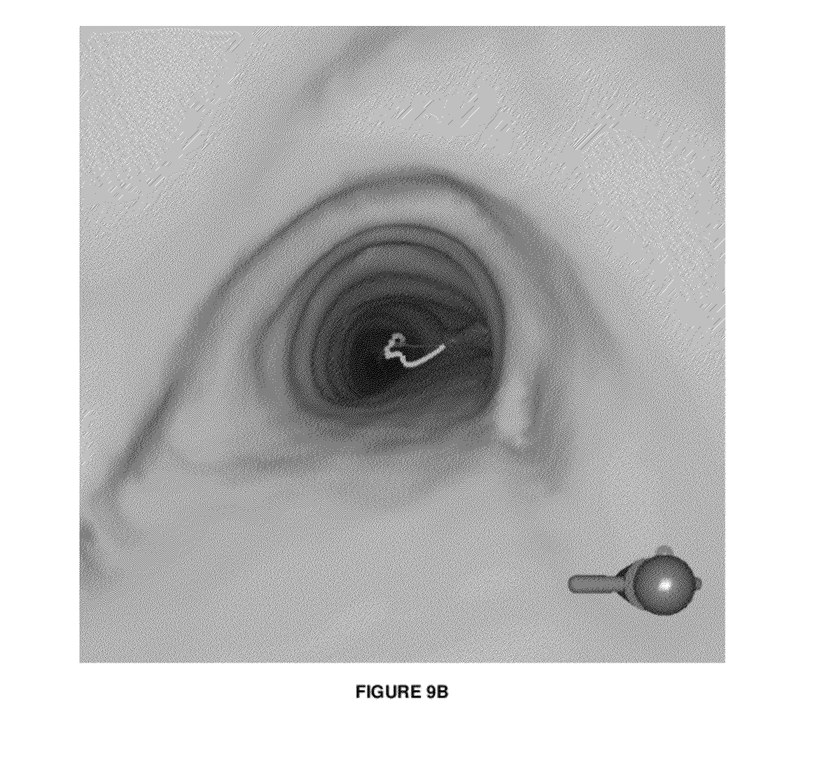

FIG. 9B shows a VB view that was generated using the centerline model when the error between the true bronchoscope location and the predicted bronchoscope location was the greatest during the phantom experiment. The error is mostly due to a poor sensor measurement that was off by 6 mm. Even with this error, guidance is still possible. Furthermore, inserting the bronchoscope to the next tape mark reduced the total Euclidean distance between the predicted location and the actual location to 5 mm (approximately the median error for the centerline bronchoscope model). The other bronchoscope models never predicted a VB location with as great an error.

The PVC-pipe experiment excluded any error from the sensor, yet it resulted in higher Euclidean distance errors on average than the phantom experiment, including the error from all method components. This is because the PVC-pipe model experiment involved navigating the bronchoscope up to a distance of 480 mm while, in the phantom experiment, the bronchoscope was only navigated up to 75 mm. Therefore, with less distance to travel, less error accumulated. Also, the path in the phantom experiment was relatively straight while the path in the PVC-pipe experiment contained 90 degree angles.

To aid the physician in staying on the correct route to the ROI, the system provides directions that are fused onto the live bronchoscope view when the virtual space and the physical space are synchronized. Assuming that a physician can follow these directions, then the two spaces will remain synchronized. Detecting if and when a physician goes off the path is possible by generating candidate views down possible branches and comparing them to the bronchoscopic video [43].

We first select candidate locations by using the above mentioned method to track the bronchoscope along two possible branches after a bifurcation, instead of just 1 route. This provides the system with two candidate bronchoscope locations. Next, we register the VB views generated from each possible branch to the live bronchoscopic video and then compare each VB view to the bronchoscopic video. This assigns a probability to each candidate view indicating if it was generated from the real bronchoscope's location. We use Bayesian inferencing techniques to combine multiple probabilities allowing the system to detect which branch the physician maneuvered the bronchoscope into in real time [43]. Near the end of either of the possible branches, the system selects the branch with the highest Bayesian inference probability as the correct branch. When the system detects that the bronchoscope is not on the optimal route to the ROI, the highlighted paths on the VB view are red instead of blue, and a traffic light indicator signals the physician to retract the bronchoscope until the physician is on the correct route.

The system invokes this branch selection algorithm every x mm of bronchoscope insertion (default x=2 mm). In between invocation of this branch selection algorithm, the system generates VB views along the branch that currently has the highest Bayesian inference. The further the bronchoscope is inserted, the more refined the Bayesian inference probability becomes. Before a view is displayed to a physician, the system can register it to the current bronchoscope video in real time using the method of Merritt et al. [26, 43].

Our method uses a sensor to measure movements made by the bronchoscope to predict where the tip of the bronchoscope is with high accuracy. This bronchoscope guidance method provides VB views that indicate where the physician is in the lungs. Encoded on these views are simple directions for the physician to follow to reach the ROI. If the physician can follow the directions, the bronchoscope will always stay on the correct path, providing continuous, real-time guidance, improving the success rate of bronchoscopic procedures. Furthermore, the system can signal the physician when they maneuver off the correct route.

This method is suited for more than just sampling ROIs during bronchoscopy. It could be useful for treatment delivery including fiducial marker planning and insertion for radiation therapy and treatment. The system, at a higher level, is suitable for thoracic surgery planning. While our system is implemented for use in the lungs, the methods presented are applicable to any application where a long thin device must be tracked along a preplanned route. Some examples include tracking a colonoscope through the colon and tracking a catheter through vasculature [7].

### REFERENCES

[1] F. Asano. Virtual bronchoscopic navigation. Clinics in Chest Medicine., 31(1):75-85, 2010.
[2] H. D. Becker and F. Herth and A. Ernst and Y. Schwarz. Bronchoscopic biopsy of peripheral lung lesions under electromagnetic guidance: A pilot study. J. Bronchology, 12(1):9-13, 2005.
[3] I. Bricault and G. Ferretti and P. Cinquin. Registration of Real and CT-Derived Virtual Bronchoscopic Images to Assist Transbronchial Biopsy. IEEE Transactions on Medical Imaging, 17(5):703-714, 1998.
[4] V. Chechani. Bronchoscopic Diagnosis of solitary pulmonary nodules and lung masses in the absence of endobronchial abnormality. Chest, 109(3):620-625, 1996.
[5] Dalrymple, N. C. and Prasad, S. R. and Freckleton, M. W. and Chintapalli, K N. Informatics in radiology (infoRAD): introduction to the language of three-dimensional imaging with multi detector CT. Radiographics, 25(5):1409-1428, 2005.
[6] M. Y. Dolina and D. C. Cornish and S. A. Merritt and L. Rai and R. Mahraj and W. E. Higgins and R. Bascom. Interbronchoscopist variability in endobronchial path selection: a simulation study. Chest, 133(4):897-905, 2008.
[7] A. Eickhoff and J. Van Dam and R. Jakobs and V. Kudis and D. Hartmann and U. Damian and U. Weickert and D. Schilling, and J. Riemann. Computer-Assisted Colonoscopy (The NeoGuide Endoscopy System): Results of the First Human Clinical Trial “PACE Study”. 102(2):261-266, 2007.
[8] J. D. Gibbs and M. W. Graham and W. E. Higgins. 3D MDCT-based system for planning peripheral bronchoscopic procedures. Computers in Biology and Medicine, 39(3):266-279, 2009.
[9] T. R. Gildea and P. J. Mazzone and D. Karnak and M. Meziane and A. C. Mehta. Electromagnetic navigation diagnostic bronchoscopy: a prospective study. Am. J. Resp. Crit. Care Med., 174(9):982-989, 2006.
[10] M. W. Graham and J. D. Gibbs and D. C. Cornish and W. E. Higgins. Robust 3D Airway-Tree Segmentation for Image-Guided Peripheral Bronchoscopy. IEEE Trans. Medical Imaging, 29(4):982-997, 2010.
[11] W. E. Grimson and G. J. Ettinger and S. J. White and T. Lozano-Perez and W. E. Wells III and R. Kikinis. An Automatic Registration Method for Frameless Stereotaxy, Image Guided Surgery, and Enhanced Reality Visualization. IEEE Trans. Med. Imaging, 15(2):129-140, 1996.
[12] J. P. Helferty and A. J. Sherbondy and A. P. Kiraly and W. E. Higgins. Computer-based system for the virtual-endoscopic guidance of bronchoscopy. Comput. Vis. Image Underst., 108(1-2):171-187, 2007.
[13] W. E. Higgins and J. P. Helferty and K. Lu and S. A. Merritt and L. Rai and K. C. Yu. 3D CT-video fusion for image-guided bronchoscopy. Comput. Med. imaging Graph., 32(3):159-173, 2008.
[14] K. Hopper and T. Lucas and K. Gleeson and J. Stauffer and R. Bascom and D. Mauger and R. Mahraj. Transbronchial biopsy with virtual CT bronchoscopy and nodal highlighting. Radiology, 221(2):531-536, 2001.
[15] E. A. Kazerooni. High Resolution CT of the Lungs. Am. J. Roentgenology, 177(3):501-519, 2001.
[16] A. P. Kiraly and J. P. Helferty and E. A. Hoffman and G. McLennan and W. E. Higgins. 3D path planning for virtual bronchoscopy. IEEE Trans. Medical Imaging, 23(11):1365-1379, 2004.
[17] J. Kleinberg and E. Tardos. Algorithm Design. Pearson Education, Inc., Boston, Mass., USA, 2006.
[18] M. Kukuk. A Model-Based Approach to Intraoperative Guidance of Flexible Endoscopy. PhD thesis, University of Dortmund, 2002.
[19] M. Kukuk. An “optimal” k-needle placement strategy and its application to guiding transbronchial needle aspirations. Computer Aided Surgery, 9(6):261-290, 2004.
[20] Kukuk, M. An “Optimal” k-Needle Placement Strategy Given an Approximate Initial Needle Position. Medical Image Computing and Computer-Assisted Intervention—MICCAI 2003 in Lecture Notes in Computer Science, pages 116-123. Springer Berlin/Heidelberg, 2003.
[21] M. Kukuk. Modeling the internal and external constraints of a flexible endoscope for calculating its workspace: application in transbronchial needle aspiration guidance. SPIE Medical Imaging 2002: Visualization, Image-Guided Procedures, and Display, S. K. Mun (ed.), v. 4681:539-550, 2002.
[22] Kukuk, M. and Geiger, B. A Real-Time Deformable Model for Flexible Instruments Inserted into Tubular Structures. In Dohi, Takeyoshi and Kikinis, Ron, editors, Medical Image Computing and Computer-Assisted Intervention—MICCAI 2002 in Lecture Notes in Computer Science, pages 331-338. Springer Berlin/Heidelberg, 2002.
[23] M. Kukuk and B. Geiger and H. Muller. TBNA-protocols: guiding transbronchial needle aspirations without a computer in the operating room. MICCAI 2001, W. Niessen and M Viergever (eds.), vol. LNCS 2208:997-1006, 2001.
[24] H. P. McAdams and P. C. Goodman and P. Kussin. Virtual bronchoscopy for directing transbronchial needle aspiration of hilar and mediastinal lymph nodes: a pilot study. Am. J. Roentgenology, 170(5):1361-1364, 1998.
[25] S. A. Merritt and J. D. Gibbs and K. C. Yu and V. Patel and L. Rai and D. C. Cornish and R. Bascom and W. E. Higgins. Real-Time Image-Guided Bronchoscopy for Peripheral Lung Lesions: A Phantom Study. Chest, 134(5):1017-1026, 2008.
[26] S. A. Merritt and L. Rai and W. E. Higgins. Real-time CT-video registration for continuous endoscopic guidance. In A. Manduca and A. A. Amini, editors, SPIE Medical Imaging 2006: Physiology, Function, and Structure from Medical Images, pages 370-384, 2006.
[27] D. Mirota and H. Wang and R. H. Taylor and M. Ishii and G. D. Hager. Toward Video-Based Navigation for Endoscopic Endonasal Skull Base Surgery. MICCAI, pages 91-99, 2009.
[28] K. Mori and D. Deguchi and K. Akiyama and T. Kitasaka and C. R. Maurer and Y. Suenaga and H. Takabatake and M. Mod and H. Natori. Hybrid bronchoscope tracking using a magnetic tracking sensor and image registration. In J. Duncan and G. Gerig, editors, Medical Image Computing and Computer Assisted Intervention 2005, pages 543-550, 2005.
[29] K. Mod and D. Deguchi and J. Hasegawa and H. Natori et al. A method for tracking the camera motion of real endoscope by epipolar geometry analysis and virtual endoscopy system. In W. Niessen and M. Viergever, editors, MICCAI 2001, pages 1-8, 2001.
[30] K. Mod and K. Ishitani and D. Deguchi and T. Kitasaka and Y. Suenaga and H. Takabatake and M. Mod and H. Natori. Compensation of electromagnetic tracking system using an optical tracker and its application to bronchoscopy navigation system. In Kevin R. Cleary and Michael I. Miga, editors, Medical Imaging 2007: Visualization and Image-Guided Procedures, number 1, pages 65090M, 2007.
[31] D. Osborne and P. Vock and J. Godwin and P. Silverman. CT identification of bronchopulmonary segments: 50 normal subjects. AJR, 142(1):47-52, 1984.
[32] Y. Sato and M. Nakamoto and Y. Tamaki and T. Sasama and I. Sakita and Y. Nakajima and M. Monden and S. Tamura. Image guidance of breast cancer surgery using 3-D ultrasound images and augmented reality visualization. IEEE Trans. on Medical Imaging, 17(5):681-693, 1998.
[33] Schwarz, Y and Greif, J and Becker, H D and Ernst, A. and Mehta, A. Real-time electromagnetic navigation bronchoscopy to peripheral lung lesions using overlaid CT images: the first human study. Chest, 129(4):988-994, 2006.
[34] Shinagawa, N. and Yamazaki, K. and Onodera, Y. and Miyasaka, K. and Kikuchi, E. and Dosaka-Akita, H. and Nishimura, M. CT-guided transbronchial biopsy using an ultrathin bronchoscope with virtual bronchoscopic navigation. Chest, 125(3):1138-1143, 2004.
[35] S. B. Solomon and P. White, Jr. and C. M. Wiener and J. B. Orens and K. P. Wang. Three-dimensionsal CT-guided bronchoscopy with a real-time electromagnetic position sensor: a comparison of two image registration methods. Chest, 118(6):1783-1787, 2000.
[36] Soper, T. D. and Haynor, D. R. and Glenny, R. W. and Seibel, E. J. Validation of CT-video registration for guiding a novel ultrathin bronchoscope to peripheral lung nodules using electromagnetic tracking. SPIE Medical Imaging, 2009.
[37] J. D. Stefansic and A. J. Herline and Y. Shyr and W. C. Chapman and J. M. Fitzpatrick and B. M. Dawant and R. L. Galloway Jr. Registration of physical space to laparoscopic Image space for use in minimally invasive hepatic surgery. IEEE Trans. Med. Imaging, 19(10):1012-1023, 2000.
[38] J. Ueno and T. Murase and K. Yoneda and T. Tsujikawa and S. Sakiyama and K. Kondoh. Three-dimensional imaging of thoracic diseases with multi-detector row CT. J. Med. Invest., 51(3-4):163-170, 2004.
[39] K. P. Wang and A. C. Mehta and J. F. Turner, eds. Flexible Bronchoscopy. Blackwell Publishing, Cambridge, Mass., 2 edition, 2003.
[40] I. Wegner, J. Biederer, R. Tetzlaff, I. Wolf, and H. P. Meinzer, “Evaluation and extension of a navigation system for bronchoscopy inside human lungs,” In Cleary, Kevin R. and Miga, Michael I., editors, SPIE Medical Imaging 2007: Visualization and Image-Guided Procedures, pages 65091H1-65091H12, 2007.
[41] K. C. Yu and E. L. Ritman and W. E. Higgins. 3D Model-Based Vasculature Analysis Using Differential Geometry. IEEE Int. Symp. on Biomedical Imaging,:177-180, 2004.
[42] K. C. Yu and E. L. Ritman and W. E. Higgins. System for the Analysis and Visualization of Large 3D Anatomical Trees. Comput Biol Med, 37(12):1802-1820, 2007.
[43] D. C. Cornish and W. E. Higgins. Bronchoscopy Guidance System Based on Bronchoscope-Motion Measurements. SPIE Medical Imaging 2012: Image-Guided Procedures, Robotic Interventions, and Modeling. To appear 2012.

1. A method of determining the location of an endoscope within a body lumen, comprising the steps of:
precomputing a virtual model of an endoscope that approximates insertion depths at a plurality of view sites along a predefined path to a region of interest (ROI);
providing an endoscope with a device operative to observe actual insertion depths during a live procedure;
comparing, in real time, the observed insertion depths to the precomputed insertion depths at each view site along the predefined path;
predicting the location of the endoscope relative to the virtual model at each view site by selecting the view site with the precomputed insertion depth that is closest to the observed insertion depth; and
generating an endoluminal rendering providing navigational instructions based upon the predicted locations.

2. The method of claim 1, wherein:
the lumen forms part of an airway tree; and
the endoscope is a bronchoscope.

3. The method of claim 1, wherein:
the device is operative to observe roll angle in addition to insertion depth; and
the observed roll angle is used to rotate the default viewing direction at a selected view site.

4. The method of claim 1, including the step of using the method of Gibbs et al. to predetermine the optimal path leading to an ROI.

5. The method of claim 1, including the step of displaying the rendered predicted locations and actual view sites from the device.

6. The method of claim 1, wherein the virtual model is a MDCT image-based shape model.

7. The method of claim 1, wherein the step of precomputing allows for an inverse lookup of the predicted locations.

8. The method of claim 1, including the step of calculating separate insertion depths to each view site along the medial axes of the lumen.

9. The method of claim 1, including the step of approximating the endoscope as a series of line segments.

10. The method of claim 1, wherein the lumen is defined using voxel locations, the method including the step of calculating separate insertion depths to any voxel location within the lumen.

11. The method of claim 1, wherein the lumen is defined using voxel locations, the method including the step of approximating the shape of the endoscope to any voxel location within the lumen.

12. The method of claim 8, wherein the insertion depth to each view site is calculated by summing distances along the lumen medial axes.

13. The method of claim 10, wherein the insertion depth to each voxel location within the lumen is calculated by finding the shortest distance from a root voxel location to every voxel location within the lumen using Dijkstra's algorithm.

14. The method of claim 10, wherein the insertion depth to each voxel location within the lumen is calculated by using a dynamic programming algorithm.

15. The method of claim 9, wherein the shape of the endoscope is approximated using the lumen medial axes.

16. The method of claim 11, wherein the shape of the endoscope to any voxel location is approximated using Dijkstra's algorithm.

17. The method of claim 11, wherein the shape of the endoscope to any voxel location is approximated using a dynamic programming algorithm.

18. The method of claim 16, wherein the edge weight used in Dijkstra's algorithm is determined using a dot product and the Euclidean distance between voxel locations within the lumen.

19. The method of claim 14, wherein the dynamic programming function includes an optimization function, and the optimization function is based on the dot product between voxel locations within the lumen.

20. The method of claim 1, wherein the device is an optical sensor.

21. A method for guiding an endoscope within a body lumen, comprising the steps of:
computing the optimal route leading to a region of interest (ROI);
tracking the tip of the endoscope;
generating an endoluminal rendering providing navigational instructions based upon the tracked locations; and
instructing a user to retract the endoscope if the endoluminal rendering indicates that the user is off the optimal route.

22. The method of claim 21, wherein:
the lumen forms part of an airway tree; and
the endoscope is a bronchoscope.

23. The method of claim 21, wherein the optimal route leading to the ROI is computed using the method of Gibbs et al.

24. The method of claim 21, wherein the method used for tracking applies the method of claim 1 to possible candidate branches based on the endoscopic insertion depth.

25. The method of claim 24, including the steps of:
registering candidate virtual bronchoscopic (VB) views to the endoscopic video; and
comparing the registered views to the endoscopic video using an image similarity metric.

26. The method of claim 25, wherein the registration of VB views to endoscopic video uses the method of Merritt et al.

27. The method of claim 25, wherein the image similarity metric is normalized sum-of-squared error.

28. The method of claim 24, including the step of creating a probability indicating if a candidate view was generated from the same location and orientation as the real bronchoscope.

29. The method of claim 28, including the step of combining multiple probabilities to make a final decision regarding which branch the endoscope actually entered.

30. The method of claim 21, including the step of displaying a view from the endoscope's tracked location and orientation that is fused with guidance information indicating if the endoscope operator is on the correct route to the ROI.

31. The method of claim 21, including the step of instructing a user to retract the endoscope if the endoscope goes off of the optimal route.
